(19) United States
(12) Patent Application Publication
(10) Pub. No.: US 2025/0249253 A1
(43) Pub. Date: Aug. 7, 2025

(54) EVOKED RESPONSE SCANNING WITH ERROR CORRECTION

(71) Applicant: Boston Scientific Neuromodulation Corporation, Valencia, CA (US)

(72) Inventors: G. Karl Steinke, Valencia, CA (US); Andrew James Haddock, Los Angeles, CA (US); Soroush Niketeghad, Stevenson Ranch, CA (US)

(21) Appl. No.: 19/024,630

(22) Filed: Jan. 16, 2025

Related U.S. Application Data
(60) Provisional application No. 63/549,976, filed on Feb. 5, 2024.

(51) Int. Cl. A61N 1/36

(52) U.S. Cl. CPC A61N 1/36135; A61N 1/36142; A61N 1/36178

(57) ABSTRACT

Systems and methods for collecting evoked responses (ERs), and using the same to guide neuromodulation are disclosed. An exemplary system includes an electrostimulator to provide electrostimulation to a neural target, a sensing circuit to sense ERs, and a controller circuit to collect first ERs sensed from a group of sensing electrodes in response to electrostimulation through first one or more stimulating electrodes. The controller circuit performs a quality check of the first ERs, determine or update an ER sampling routine including timings or an order of performing multiple stimulation and ER collection tests, and sequentially execute the multiple stimulation and ER collection tests, and collect second ERs according to the determined or updated ER sampling routine. When the second ERs satisfy an acceptance criterion, a recommendation can be provided to a user to reposition the lead or to set or adjust a stimulation setting.

### CLAIM OF PRIORITY

This application claims the benefit of U.S. Provisional Application No. 63/549,976, filed on Feb. 5, 2024, which is hereby incorporated by reference in its entirety.

### TECHNICAL FIELD

This document relates generally to medical systems, and more particularly, but not by way of limitation, to systems, devices, and methods for collecting evoked responses and using the same to guide neuromodulation therapy.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc. A neurostimulation device (e.g., DBS, SCS, PNS or TENS) may be configured to treat pain. By way of example and not limitation, a DBS system may be configured to treat tremor, bradykinesia, and dyskinesia and other motor disorders associated with Parkinson's Disease (PD).

It has been proposed to use evoked potentials (also referred to as evoked responses) to guide neurostimulation therapy. For example, Evoked Resonant Neural Activity (ERNA) has been proposed as a feedback signal for subthalamic nucleus (STN) DBS therapy for Parkinson's disease. ERNA may also be referred to by other names such as DBS Local Evoked Potentials (DLEP), Evoked oscillatory neural responses (EONR), and other terms. Evoked potentials, including ERNA, may be present in other indications and anatomical structures or locations.

Evoked responses (ERs) are ideally representative of physiological responses of neural tissue to stimulation. However, ERs may be affected by various confounding factors. Inaccurate ER measurements may affect decision marking with regard to stimulation site selection, stimulation setting, and lead placement, which may lead to suboptimal neurostimulation therapy.

### SUMMARY

Desired evoked response targets (e.g., ERNA-based targets for DBS, such as STN DBS or GPi DBS) vary from patient to patient depending on anatomical target locations, trajectory of the lead placement, and evoking and recording settings. To accommodate variations in desired evoked response targets, multiple stimulation-ER collection tests may be performed where a large volume of ER recordings are collected from multiple anatomical locations via respective sensing electrodes (also referred to as recording electrodes) in response to evoking stimulation through one or a combination of a group of stimulating electrodes. The resulting ERs may be analyzed to decide if the ERs or characteristics or features derived therefrom satisfy an acceptance criterion, such as matching a desired or target ER characteristic. This process is referred to as a spatial survey of stimulating sites. Desired or target ERs corresponds to a desired stimulation effect, such as a maximal or desired therapeutic benefits (e.g., reduced symptoms) and/or minimal side effects. The finding of the desired or target ERs may be used to guide lead placement (e.g., pushing, pulling, or rotating the lead in a given trajectory, or shifting the lead to a new trajectory) and optimize stimulation site and stimulation setting to improve therapy efficacy.

For an accurate decision-making with regard to an optimal or desired stimulation site and stimulation setting, the ERs sensed during the spatial survey are generally required to truly represent tissue physiological responses to stimulation, and are not or least affected by other confounding factors. Ideally, ERs are sensed under substantially stable and consistent patient conditions and testing conditions (e.g., consistent ER sensing electrodes and sensing circuitry configuration) during the spatial survey process. In practice, however, patient conditions and/or testing conditions may vary. For example, in a spatial survey to optimize DBS, patient medication, therapy, anesthesia and arousal state, among other factors, may affect the patient's brain state. History and magnitude of stimulation presented, including the spatial survey evoking stimulation, may also affect the brain state and the resultant survey response. Evoking stimulation applied at one stimulation site may produce “residual effects” at sensing locations particularly those proximate to that stimulation site, thereby causing perturbations on ERs subsequently sensed in response to stimulation at another stimulation site. Such residual effects may be due to a spatial overlap between stimulation fields produced by stimulations at adjacent stimulation sites. The residual effects may also be contributed by an overlap between distinct spatial or anatomical structures; i.e., a neural-circuitry overlap. For example, in DBS, brain structures such as STN, globus pallidus internus (GPi), and globus pallidus externus (GPe) are interconnected via neural pathways, and may all be evoked or affected concurrently by stimulation at a particular stimulation site. Each time an anatomical target or neural circuitry is engaged by stimulation, it is perturbed. The perturbation can move the anatomical target out of its initial state, thereby affecting the ERs sensed therefrom.

Testing conditions, such as ER sensing equipment conditions, may also vary during the spatial survey. For example, ER sensing electrodes placed at different sensing locations of the brain may have different tissue-contacting surface areas and thus different electrode-tissue conductance and tissue conductance. ER sensing circuitry (e.g., sense amplifiers and other circuitry components) may vary from one sense channel to another. The conditions of the sensing circuitry may vary from one sense channel to another, or from one evoking stimulation pulse or sensing window measurement to another.

Inconsistent patient conditions and/or testing equipment conditions (such as different sense electrodes and/or different sense channels) during spatial survey may reduce the validity and usefulness of the ERs collected under such conditions during the spatial survey process. Embodiments of the present subject matter provide improved ER sampling strategies during spatial survey that can avoid, minimize, or correct perturbations introduced by confounding factors such as inconsistent patient conditions and/or testing equipment conditions. An exemplary neuromodulation system includes an electrostimulator to provide electrostimulation to a neural target via one or more stimulating electrodes on at least one lead, a sensing circuit to sense evoked responses (ERs) via one or more sensing electrodes on the at least one lead, and a controller circuit to collect first ERs sensed from a group of sensing electrodes in response to electrostimulation through first one or more stimulating electrodes. The controller circuit can perform a quality check of the first ERs, determine or update an ER sampling routine including timings or an order of performing multiple stimulation and ER collection tests, and sequentially execute the multiple stimulation and ER collection tests and collect second ERs according to the determined or updated ER sampling routine. When the second ERs satisfy an acceptance criterion, a recommendation can be provided to a user to reposition the lead or to set or adjust a stimulation setting.

Example 1 is a neuromodulation system that includes: an electrostimulator configured to provide electrostimulation to a neural target of a patient via one or more stimulating electrodes on at least one lead; a sensing circuit configured to sense evoked responses (ERs) to electrostimulation via one or more sensing electrodes on the at least one lead; and a controller circuit operably connected to the electrostimulator and the sensing circuit, the controller circuit configured to: in response to electrostimulation of the neural target through first one or more stimulating electrodes in accordance with a stimulation setting, collect first ERs sensed from a group of sensing electrodes on the at least one lead; perform a quality check of the first ERs; based on a result of the quality check of the first ERs, determine or update an ER sampling routine including one or more of timings or an order of performing multiple stimulation and ER collection tests subsequent to the electrostimulation through the first one or more stimulating electrodes; and sequentially execute the multiple stimulation and ER collection tests and collect second ERs in accordance with the determined or updated ER sampling routine.

In Example 2, the subject matter of Example 1 optionally includes the controller circuit that can be configured to, when the second ERs satisfy an acceptance criterion, provide a recommendation to a user to reposition the at least one lead or to set or adjust the stimulation setting.

In Example 3, the subject matter of any one or more of Examples 1-2 optionally includes the controller circuit that can perform the quality check including to determine that the first ERs pass the quality check when each of the first ERs falls within a specific ER value range, or fail the quality check when one or more of the ERs fall outside of the specific ER value range.

In Example 4, the subject matter of Example 3 optionally includes the controller circuit that can be configured to determine the specific ER value range based on a statistical distribution model of the first ERs.

In Example 5, the subject matter of any one or more of Examples 3-4 optionally includes, when one or more of the first ERs fail the quality check, the controller circuit that can be configured to determine or update the ER sampling routine including a post-stimulation time delay before initiating a subsequent stimulation and ER collection test involving stimulation through second one or more stimulating electrodes different than the first one or more stimulating electrodes, wherein the post-stimulation time delay is inversely related to a spatial distance between the first and the second stimulating electrodes.

In Example 6, the subject matter of any one or more of Examples 3-5 optionally includes, when one or more of the first ERs fail the quality check, the controller circuit that can be configured to determine or update the ER sampling routine including an order of executing the subsequent multiple stimulation and ER collection tests involving stimulations via respective stimulating electrodes different than the first one or more stimulating electrodes, wherein the order of executing the subsequent multiple stimulation and ER collection tests is inversely related to spatial distances between the respective stimulating electrodes and the first one or more stimulating electrodes.

In Example 7, the subject matter of any one or more of Examples 1-6 optionally includes the electrostimulation of the neural target that can include a plurality of stimulation bursts, and the first ERs include temporal ER portions corresponding to the plurality of stimulation bursts, wherein the controller circuit is configured to perform the quality check of the first ERs at one or more distinct time scales with respect to the electrostimulation, including at least one of: a first quality check of one of the temporal ER portions corresponding to an individual burst of the plurality of stimulation bursts; or a second quality check of a plurality of the temporal ER portions corresponding to multiple distinct bursts of the plurality of stimulation bursts.

In Example 8, the subject matter of any one or more of Examples 1-7 optionally includes the controller circuit that can be configured to: perform the quality check of the second ERs; identify, from the multiple stimulation and ER collection tests, at least one failed test with corresponding one or more ERs that fail the quality check; and repeat the identified at least one failed test up to a specific maximum number of attempts.

In Example 9, the subject matter of Example 8 optionally includes the controller circuit that can be configured to repeat the identified at least one test at a scheduled time, including: to repeat the identified at least one failed test prior to executing any other of the multiple stimulation and ER collection tests; or to append the identified at least one failed test to the end of the multiple stimulation and ER collection tests, and repeat the identified at least one test after executing every other of the multiple stimulation and ER collection tests.

In Example 10, the subject matter of any one or more of Examples 1-9 optionally includes a programming device and an ambulatory medical device (AMD) communicatively coupled to the programming device, the AMD including at least a portion of the controller circuit configured to sequentially execute the multiple stimulation and ER collection tests and collect the second ERs.

In Example 11, the subject matter of any one or more of Examples 1-10 optionally includes a programming device and an ambulatory medical device (AMD) communicatively coupled to the programming device, the programming device including at least a portion of the controller circuit configured to sequentially execute the multiple stimulation and ER collection tests and collect the second ERs.

In Example 12, the subject matter of any one or more of Examples 1-11 optionally includes the controller circuit that can be further configured to: track changes in patient health or medical conditions and identify a period of substantially consistent condition; and collect ERs during the identified period of substantially consistent condition.

In Example 13, the subject matter of any one or more of Examples 1-12 optionally includes the sensing circuit that can include a dedicated sensing channel electrically coupled to two or more sensing electrodes on the lead to concurrently sense ERs therefrom in response to electrostimulation through each of two or more distinct stimulation electrodes.

In Example 14, the subject matter of Example 13 optionally includes the two or more sensing electrodes that can be at least temporarily electrically shorted to each other when coupled to the dedicated sensing channel, the dedicated sensing channel configured to sense the ERs from the electrically shorted electrodes.

In Example 15, the subject matter of any one or more of Examples 13-14 optionally includes two or more sensing electrodes electrically coupled to the dedicated sensing channel that can include two or more segmented electrodes at a specific longitudinal level of the lead.

Example 16 is a method of providing neurostimulation to a neural target of a patient via a neuromodulation system that comprises an electrostimulator and at least one lead coupled thereto. The method includes steps of: delivering electrostimulation to the neural target in accordance with a stimulation setting via first one or more stimulating electrodes on the at least one lead; collecting first evoked responses (ERs) from each of a group of sensing electrodes on the at least one lead via a sensing circuit; performing a quality check of the first ERs; based on a result of the quality check of the first ERs, determining or updating an ER sampling routine including one or more of timings or an order of performing multiple stimulation and ER collection tests subsequent to the electrostimulation through the first one or more stimulating electrodes; sequentially executing the multiple stimulation and ER collection tests and collecting second ERs in accordance with the determined or updated ER sampling routine; and when the second ERs satisfy an acceptance criterion, providing a recommendation to a user to reposition the at least one lead or to set or adjust the stimulation setting.

In Example 17, the subject matter of Example 16 optionally includes determining an ER value range based on a statistical distribution model of the first ERs, wherein performing the quality check includes determining that the first ERs pass the quality check when each of the first ERs falls within the determined ER value range, or fail the quality check when one or more of the ERs fall outside of the determined ER value range.

In Example 18, the subject matter of Example 17 optionally includes determining or updating the ER sampling routine that can include, when one or more of the first ERs fail the quality check, determining a post-stimulation time delay before initiating a subsequent multiple stimulation and ER collection test involving stimulation through second one or more stimulating electrodes different than the first one or more stimulating electrodes, the post-stimulation time delay inversely related to a spatial distance between the first and the second stimulating electrodes.

In Example 19, the subject matter of any one or more of Examples 17-18 optionally includes determining or updating the ER sampling routine that can include, when one or more of the first ERs fail the quality check, determine or update an order of executing the subsequent multiple stimulation and ER collection tests involving stimulations via respective stimulating electrodes different than the first one or more stimulating electrodes, the order of executing the subsequent multiple stimulation and ER collection tests inversely related to spatial distances between the respective stimulating electrodes and the first one or more stimulating electrodes.

In Example 20, the subject matter of any one or more of Examples 16-19 optionally includes: performing the quality check of the second ERs; identifying, from the multiple stimulation and ER collection tests, at least one failed test with corresponding one or more ERs that fail the quality check; and repeating the identified at least one failed test up to a specific maximum number of attempts.

In Example 21, the subject matter of Example 20 optionally includes repeating the identified at least one test that can occur at a scheduled time prior to executing any other of the multiple stimulate on and ER collection tests, or after executing every other of the multiple stimulation and ER collection tests.

In Example 22, the subject matter of any one or more of Examples 16-21optionally includes the electrostimulation of the neural target that can include a plurality of stimulation bursts, and the first ERs include temporal ER portions corresponding to the plurality of stimulation bursts, wherein the quality check of the first ERs is performed at one or more distinct time scales with respect to the electrostimulation, including at least one of: a first quality check of one of the temporal ER portions corresponding to an individual burst of the plurality of stimulation bursts; or a second quality check of a plurality of the temporal ER portions corresponding to multiple distinct bursts of the plurality of stimulation bursts.

In Example 23, the subject matter of any one or more of Examples 16-22 optionally includes the first or the second ERs that can include ERs to electrostimulation through each of two or more distinct stimulation electrodes, and sensed from two or more sensing electrodes at least temporarily electrically shorted to each other and coupled to a dedicated sensing channel.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrostimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrostimulation system.

FIG. 5 illustrates, by way of example, an example of an electrical therapy-delivery system.

FIG. 6 illustrates, by way of example and not limitation, a monitoring system and/or the electrical therapy-delivery system of FIG. 5, implemented using an implantable medical device (IMD).

FIG. 7 illustrates an example of a neuromodulation system configured to provide ER-guided implantation (e.g., lead placement) and neuromodulation therapy such as DBS.

FIGS. 8-10 illustrate examples of stimulating-sensing electrode configurations that may be used in a spatial survey.

FIG. 11 illustrates an example of overlapping stimulation fields produced by stimulations through different stimulating electrodes on a lead.

FIG. 12 illustrates an example of anatomical overlap between neural structures in a parasagittal slice through the nonhuman primate brain.

FIGS. 13A-13B illustrate examples of Gaussian models of ERs sensed from respective sensing electrodes and quality check of the ERs based on model parameters.

FIGS. 14A-14B illustrate examples of ER quality check at different time scales with respect to the evoking electrostimulation and corresponding corrective actions.

FIG. 15 illustrates an example of stimulation fields produced by stimulation-ER collection tests sequentially executed during a spatial survey process.

FIG. 16 is a flowchart illustrating an example method of collecting ERs and providing ER-based neurostimulation to a neural target of a patient.

FIG. 17 illustrates generally a block diagram of an example machine upon which any one or more of the techniques (e.g., methodologies) discussed herein may perform.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Evoked responses (ERs) may be used to guide lead implantation and placement and programming of neuromodulation therapy. The ERs may be produced by evoking stimulation or therapeutic stimulation delivered through one or more stimulating electrodes on at least one lead. Stimulation may be located (1) where placing evoking pulses gets a desired response such as to maximize ERNA, (2) where listening for responses gets a desired response (e.g., maximize ERNA, (3) where placing lead is desired (e.g., best for therapy, and (4) where placing stimulation on the lead is desired (e.g., maximize therapy and/or minimize/counter side effects). The ERs may be modulated by factors including, for example, amplifier settings, relationships between stimulating and sensing electrodes, natures of stimulating or sensing electrodes including geometry and surface among other factors, and signal processing occurring within analog or digital hardware, firmware, or software during and after measurement including treatment. The target may be an anatomical target such as like a volume (e.g., STN), a collection of fibers, a sub-region (motor STN, or dorsolateral STN), a volume of interest described within or related to a particular patient's brain such as from atlas or aggregate prior information, or a “point” that may be described by optimizing a stimulation location (e.g., any one of (1)-(4) above).

Various embodiments described in this document implement improved ER sampling strategies during a spatial survey, which can help avoid, minimize, or correct perturbations such as due to inconsistent patient conditions or testing equipment conditions, among other confounding factors. The presence and the degree of perturbation and the validity of the ERs collected may be assessed using a statistical model. In one embodiment, an improved ER sampling routine includes determining or adjusting a post-stimulation time delay for initiating a subsequent stimulation and ER collection test involving a different stimulating electrode. In another embodiment, an improved ER sampling routine includes determining or adjusting an order of executing multiple stimulation and ER collection tests each involving stimulation through a corresponding stimulating electrode. In yet another embodiment, an improved ER sampling routine includes tracking changes in patient health or medical conditions determining a period of substantially consistent condition, and collecting ERs during the period of substantially consistent condition, In a further embodiment, an improved ER sampling routine includes sensing ERs concurrently via a specified set of sensing electrodes (at respective sensing locations) through a dedicated sense channel. The ERs collected according to any of such improved sampling routines, or features derived therefrom, may be evaluated against an acceptance criterion. Once satisfying the acceptance criterion, the ERs or the extracted ER features may be used in guiding lead placement or device programming. The improved ER sampling strategies as described in the present document may help reduce or minimize perturbations to ER due to varying patient conditions, residual effects on sensing locations, and inconsistent testing equipment conditions, increase ER quality and usability, and therefore improve effectiveness and efficiency of ER-based lead placement and therapy programming.

This disclosure refers to ERNA-based targets for DBS, such as may be used to treat Parkinson's Disease, as a nonlimiting example of an ER to electrostimulation provided by an electrostimulator. The present subject matter may also be applied for other ERs to other electrostimulation. The electrostimulation may be therapeutic in nature in some examples, or diagnostic in nature in others.

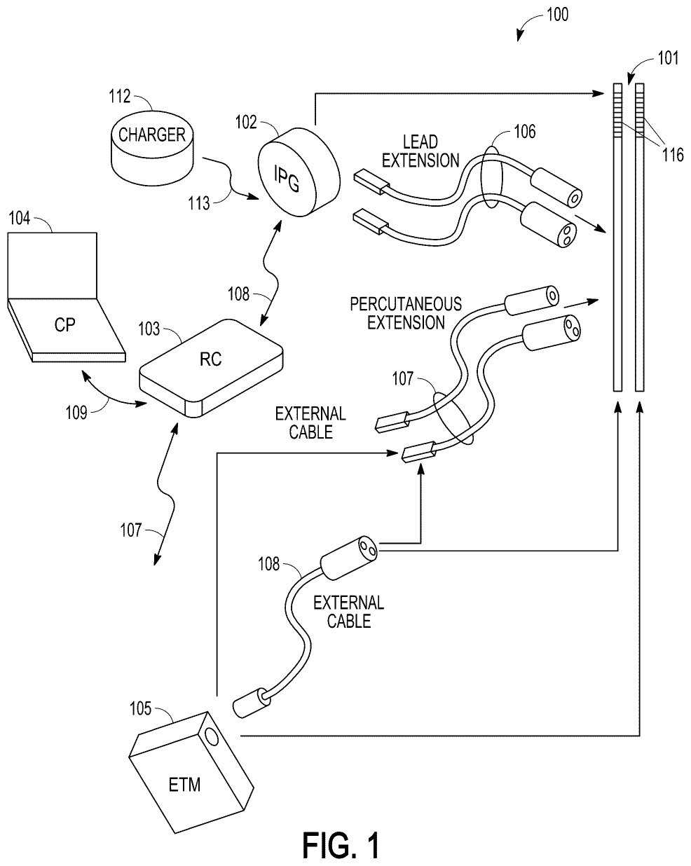

FIG. 1 illustrates, by way of example and not limitation, an electrostimulation system 100, which may be used to deliver DBS. The electrostimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Alternatively, a surgical paddle lead can be used in place of one or more of the percutaneous leads. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneous charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

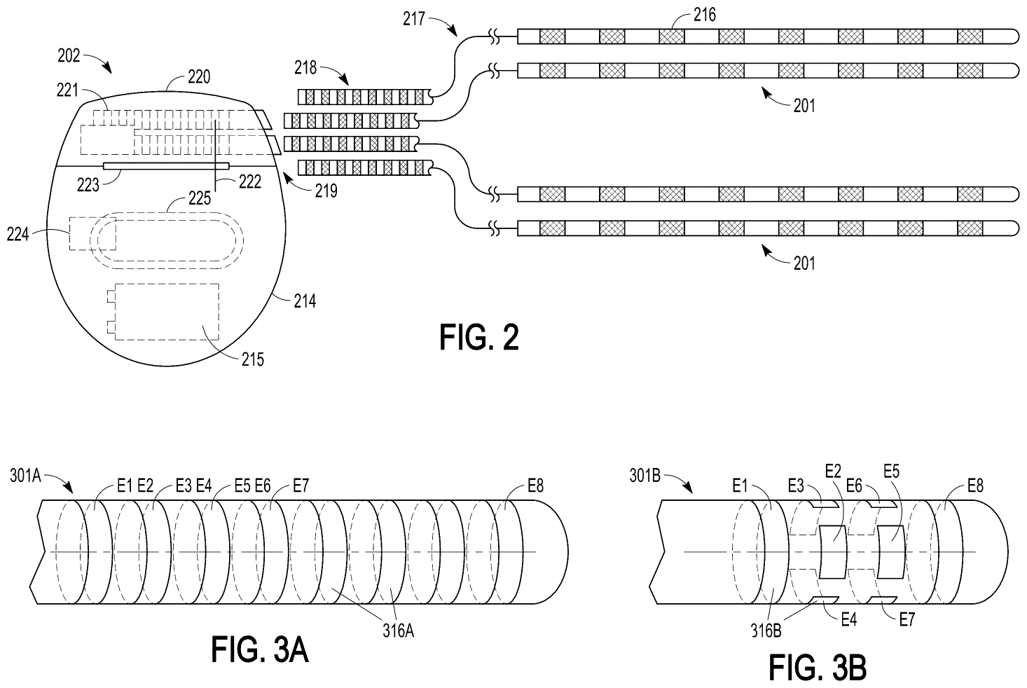

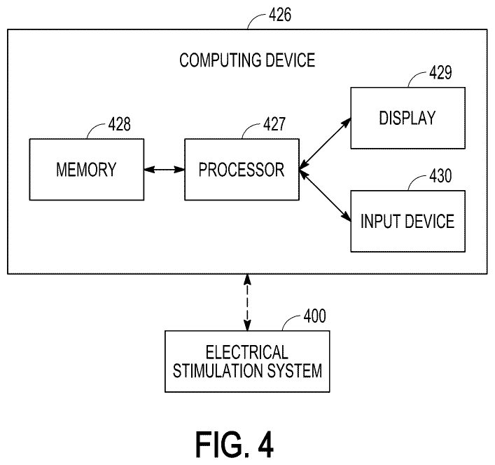

FIG. 2 illustrates, by way of example and not limitation, an IPG 202 in a DBS system. The IPG 202, which is an example of the IPG 102 of the electrostimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201 can be rotatable so that the electrodes 216 can be aligned with the target neurons after the neurons have been located such as based on the recorded signals. The electrodes 216 can include one or more ring electrodes, and/or one or more rows of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a radiofrequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) can be tunneled through and under the neck and the scalp, with the electrodes 216 implanted through holes drilled in the skull and positioned for example in the subthalamic nucleus (STN) in each brain hemisphere. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulating electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into rows of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all rows of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The rows of segmented electrodes may be positioned in irregular or regular intervals along a length of the lead 201.

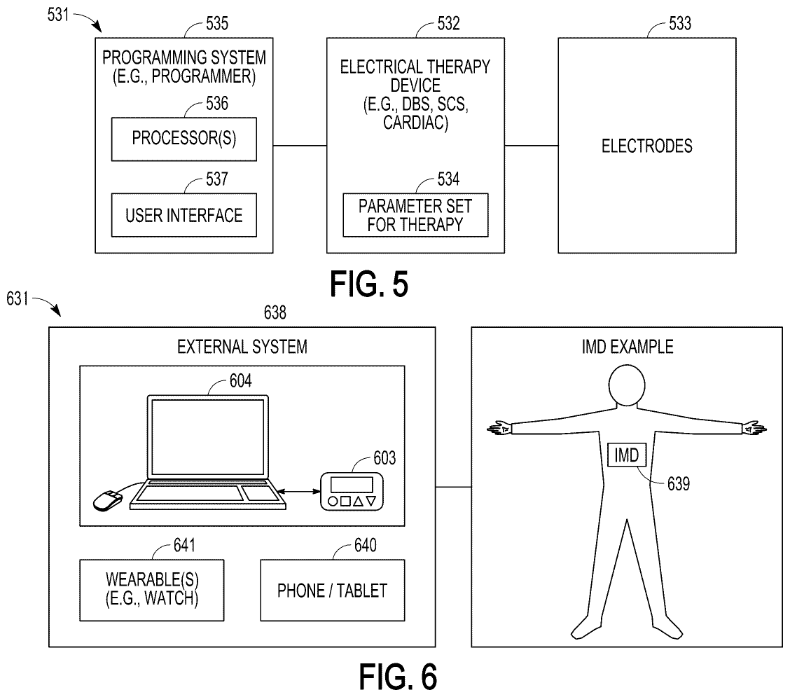

FIG. 4 illustrates, by way of example and not limitation, a computing device 426 for programming or controlling the operation of an electrostimulation system 400. The computing device 426 may include a processor 427, a memory 428, a display 429, and an input device 430. Optionally, the computing device 426 may be separate from and communicatively coupled to the electrostimulation system 400, such as system 100 in FIG. 1 Alternatively, the computing device 426 may be integrated with the electrostimulation system 100, such as part of the IPG 102, RC 103, CP 104, or ETM 105 illustrated in FIG. 1. The computing device may be used to perform process(s) for sensing parameter(s).

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrostimulation system 100, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, which is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least one the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein.

The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG. The computing device 426 may include other output(s) such as speaker(s) and haptic output(s) (e.g., vibration motor).

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

The electrostimulation system 400 may include, for example, any of the components illustrated in FIG. 1. The electrostimulation system 400 may communicate with the computing device 426 through a wired or wireless connection or, alternatively or additionally, a user can provide information between the electrostimulation system 400 and the computing device 426 using a computer-readable medium or by some other mechanism.

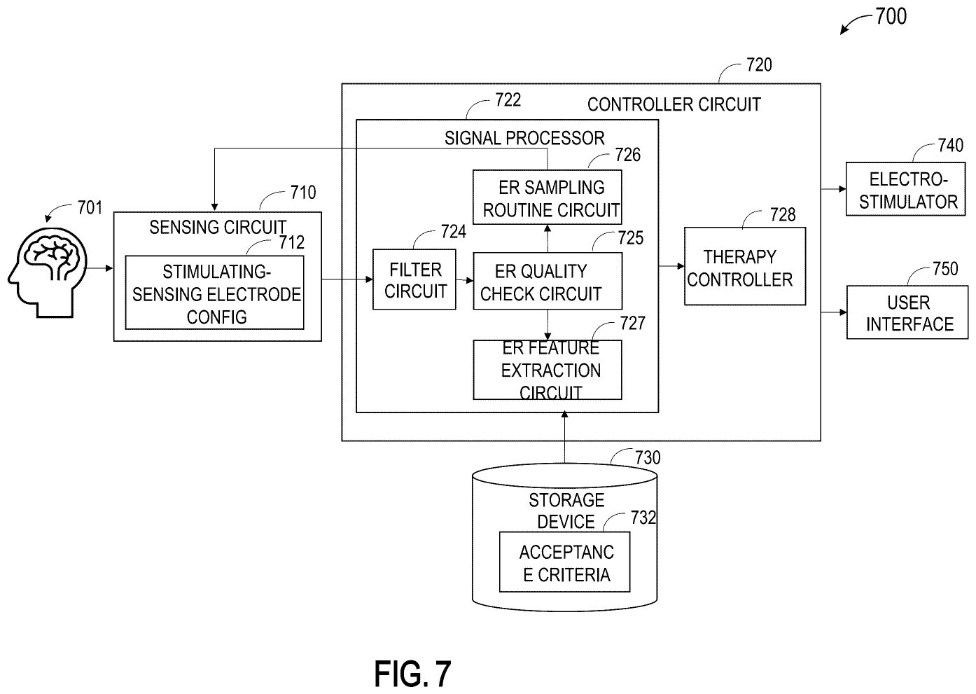

FIG. 5 illustrates, by way of example, an example of an electrical therapy-delivery system. The illustrated system 531 includes an electrical therapy device 532 configured to deliver an electrical therapy to electrodes 533 to treat a condition in accordance with a programmed parameter set 534 for the therapy. The system 531 may include a programming system 535, which may function as at least a portion of a processing system, which may include one or more processors 536 and a user interface 537. The programming system 535 may be used to program and/or evaluate the parameter set(s) used to deliver the therapy. The illustrated system 531 may be a DBS system.

In some embodiments, the illustrated system 531 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

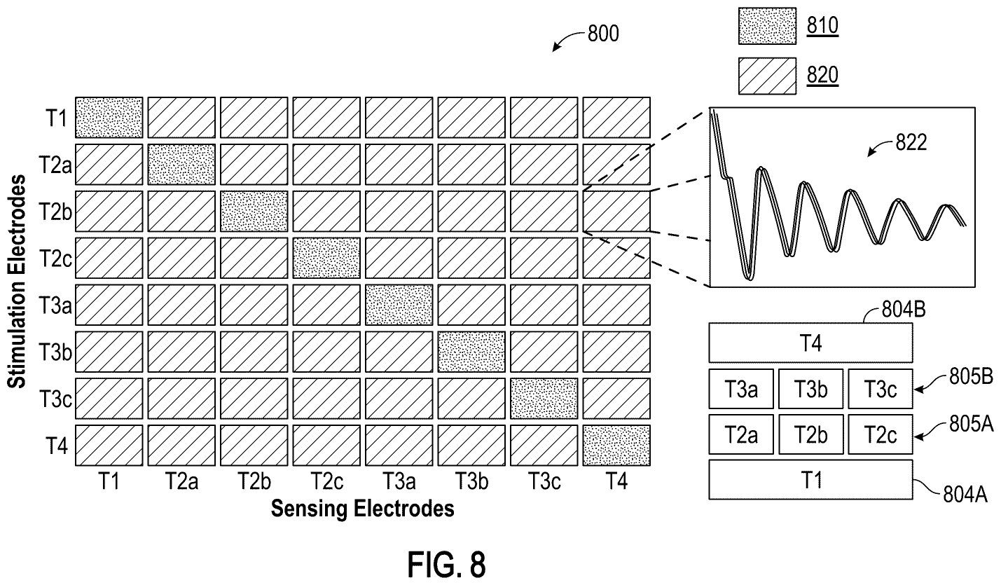

FIG. 6 illustrates, by way of example and not limitation, a monitoring system and/or the electrical therapy-delivery system of FIG. 5, implemented using an implantable medical device (IMD). The illustrated system 631 includes an external system 638 that may include at least one programming device. The illustrated external system 638 may include a clinician programmer 604, similar to CP 104 in FIG. 1, configured for use by a clinician to communicate with and program the neuromodulator, and a remote control device 603, similar to RC 103 in FIG. 1, configured for use by the patient to communicate with and program the neuromodulator. For example, the remote control device 603 may allow the patient to turn a therapy on and off, change or select programs, and/or may allow the patient to adjust patient-programmable parameter(s) of the plurality of modulation parameters. FIG. 6 illustrates an IMD 639, although the monitor and/or therapy device may be an external device such as a wearable device. The external system 638 may include a network of computers, including computer(s) remotely located from the IMD 639 that are capable of communicating via one or more communication networks with the programmer 604 and/or the remote control device 603. The remotely located computer(s) and the IMD 639 may be configured to communicate with each other via another external device such as the programmer 604 or the remote control device 603. The remote control device 603 and/or the programmer 604 may allow a user (e.g., patient and/or clinician or rep) to answer questions as part of a data collection process. The external system 638 may include personal devices such as a phone or tablet 640, wearables such as a watch 641, sensors or therapy-applying devices. The watch may include sensor(s), such as sensor(s) for detecting activity, motion and/or posture. Other wearable sensor(s) may be configured for use to detect activity, motion and/or posture of the patient. The external system 638 may include, but is not limited to, a phone and/or a tablet. Notifications may be sent to the patient, physician, device rep or other users via the external system and through remote portals (e.g., web-based portals) provided by remote systems.

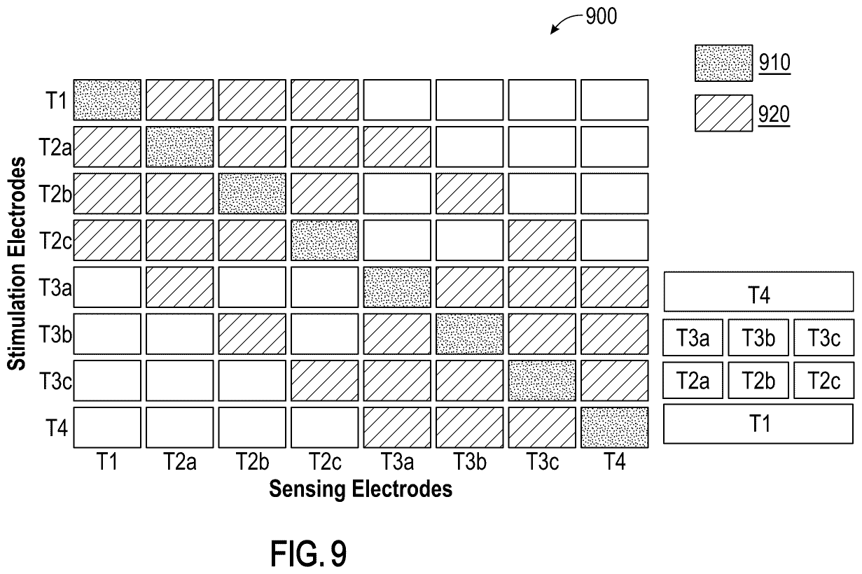

FIG. 7 illustrates an example of a neuromodulation system 700 configured to provide ER-guided lead placement and neuromodulation therapy such as DBS. The system 700 implements an improved ER sampling routine to avoid, minimize, or correct perturbation introduced by confounding factors such as inconsistent patient conditions or testing equipment conditions. The system 700 includes a sensing circuit 710, a controller circuit 720, a storage device 730, an electrostimulator 740, and a user interface 750. Portions of the system 700 may be implemented in the IPG 102 or the CP 104.

The sensing circuit 710 may be operatively connected to one or more leads and electrodes associated therewith, such as ring electrodes or segmented electrodes on the non-directional lead 301A or the directional lead 301B. The ring electrodes and/or the segmented electrodes may also be electrically coupled to the electrostimulator 740. The ring electrodes and/or the segmented electrodes may be configured as sensing electrodes for sensing ERs, or as stimulating electrodes for delivering electrostimulation pulses. The sensing circuit 710 may sense ERs from one or more sensing electrodes on a lead placed at target issue (e.g., STN) of a patient 701 in response to electrostimulation pulses delivered from a stimulating electrode at a stimulation site (e.g., a brain target).

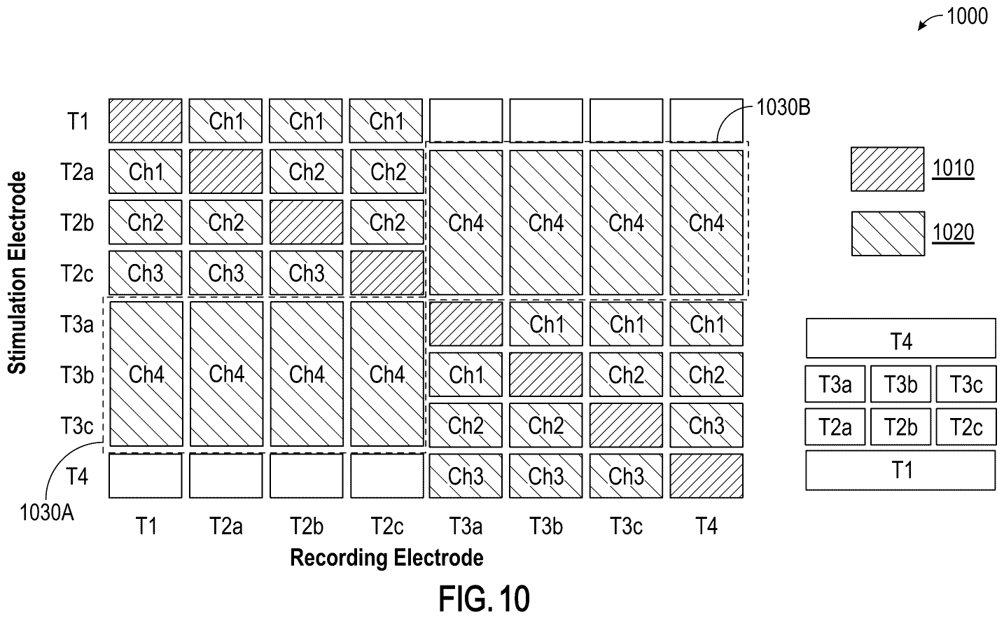

The sensing circuit 710 may sense ERs in accordance with a stimulating-sensing electrode configuration 712, such as defined in stimulation-ER collection tests to be executed during a spatial survey process with the goal of identifying an optimal or desired stimulation site and stimulation setting. During the spatial survey, multiple stimulation-ER collection tests are performed, where a large volume of ER recordings are collected in response to evoking stimulation through each of a plurality of stimulating electrodes. The resulting ERs are analyzed, and when they satisfy a specific acceptance criterion, such as matching a desired or target ER characteristic, optimal or desired stimulation site and stimulation setting may be determined accordingly. Referring to FIGS. 8-10, the diagrams therein illustrate various examples of the stimulating-sensing electrode configuration 712 that may be used in stimulation-ER collection tests during a spatial survey. In FIG. 8, the stimulating-sensing electrode configuration is represented an a two-dimensional (2D) array, graphically illustrated as an ER sensing map 800, where the sensing electrodes are indexed on the horizontal axis, and the stimulating electrodes are indexed on the vertical axis. The sensing electrodes and the stimulating electrodes are each selected from electrodes on a portion of a DBS lead. By way of example and not limitation, the electrodes include ring electrodes 804A (electrode “T1”) and 804B (electrode “T4”) and two rows of segmented electrodes 805A and 805B, all arranged in-line along the DBS lead. The two rows of segmented electrodes 805A and 805B each include three segmented electrodes (T2a, T2b, and T2c in 805A, T3a, T3b, and T3c in 805B) arranged about a circumference of the DBS lead. Other number of segmented electrodes can be included in one or more rows along the lead. In some examples, the sensing electrodes or the stimulating electrodes may be selected from electrodes not on the DBS leads. For example, at least some sensing electrodes or the stimulating electrodes may be selected from skin patch electrodes. Other nomenclatures and methods of describing the evoking and recording electrodes may be used, including those which involve multiple evoking electrodes with proportioned or fractionalized current, or Multiple Independent Current Control (MICC) to generate precise control to refine the size and shape of the stimulation field, designed to customize therapy for individual patients.

The diagonal elements in the 2D array as shown in the ER sensing map 800 represent “on-diagonal” sensing configuration where the same electrode is used for delivering electrostimulation pulses and for recording an ER to that electrostimulation in the same stimulation session. Such electrode is also referred to as “on-diagonal” sensing electrode, and the ER sensed therefrom is referred to as “on-diagonal” ER 810. The off-diagonal elements in the 2D array represent an “off-diagonal” sensing configuration where different electrodes are used for delivering electrostimulation pulses and for recording an ER to that electrostimulation in the same stimulation session. The sensing electrodes are also referred to as “off-diagonal” sensing electrodes, and the ER sensed therefrom is referred to as “off-diagonal” ER 820. The ERs may be recorded in multiple stimulation-ER recording sessions. For example, when electrostimulation pulses are delivered from electrode T1, an “on-diagonal” ER may be recorded from electrode T1, and “off-diagonal” ERs may be recorded from one or more of the rest of the electrodes (T2a-Tac, Ta3a-T3c, and T4). The stimulation-ER recording session can be repeated when stimulation is delivered from other electrodes. As an example, off-diagonal ERs 822 are recorded from sensing electrode T4 in response to stimulation delivered at electrode T2b. ERs recorded in accordance with the ER sensing map 800 can then be analyzed and used for guiding lead placement and device programming, as will be discussed further below.

Recording ERs in accordance with the full ER sensing map (i.e., to include both the “on-diagonal” and “off-diagonal” configurations) as depicted in FIG. 8 can be time consuming and take up a large amount of system sources and memory spaces. Additionally, the “on-diagonal” ERs are prone to stimulation artifacts strong enough to contaminate the ER component of interest. To avoid such effect, in an example, the sensing circuit 710 can be configured to sense ERs only from the “off-diagonal” electrodes but not from the “on-diagonal” electrodes, and only the “off-diagonal” ERs are used for guiding the lead placement and device programming.

An alternative to the “off-diagonal” stimulating-sensing configuration involves only a selected subset, less than an entirety, of available electrodes for ER sensing, as depicted in an ER sensing map 900 of FIG. 9. The selected subset can include non-diagonal electrodes within a specific proximity or with a specific geometric relationship to the stimulating electrode being used for delivering electrostimulation pulses. One such ER sensing configuration is also referred to as a “nearest neighbor” configuration. In an example, the nearest neighbor configuration includes two or more sensing electrodes immediate adjacent to the stimulating electrode on the lead. For example, in response to electrostimulation delivered at stimulating electrode T1, ERs may be sensed only from three nearest neighbor electrodes T2a, T2b, and T2c of the row of segmented electrodes 805A. In response to electrostimulation delivered at stimulating electrode T2a, ERs may be sensed only from four nearest neighbor electrodes including T2b and T2c of the row of segmented electrodes 805A, T3a of the row of segmented electrodes 805B, and the ring electrode T1. In response to electrostimulation delivered at stimulating electrode T2b, ERs may be sensed only from four nearest neighbor electrodes including T2a and T2c of the row of segmented electrodes 805A, T3b of the row of segmented electrodes 805B, and the ring electrode T1. In response to electrostimulation delivered at stimulating electrode T2c, ERs sensed only from four nearest neighbor electrodes including T2a and T2b of the row of segmented electrodes 805A, T3c of the row of segmented electrodes 805B, and the ring electrode T1. The above stimulation-ER recording sessions continue with stimulating at T3a, T3b, T3c, and T4 electrodes and recording at respective three or four nearest neighbor “non-diagonal” electrodes. In the ER sensing map 900, the ERs 920 are sensed from the “nearest neighbor” electrodes with respect to each stimulating electrode. Similar to the ER sensing map 800, the “on-diagonal” ERs 910 may be excluded from the ERs being used for guiding the lead placement and device programming.

For stimulating-sensing configurations using full off-diagonal electrodes as shown in ER sensing map 800, or partial off-diagonal electrodes such as only those “nearest neighbor” electrodes as shown ER sensing map 900, the sensing circuit 710 senses ERs from the off-diagonal electrodes using separate and distinct sensing channels during the spatial survey. As stated above, inconsistent testing equipment conditions (including, for example, different sense electrodes with different tissue-contacting surface areas and thus different tissue conductance, and different sense channel characteristics) may reduce the validity and usefulness of the ERs for the purpose of spatial survey. To reduce or minimize the perturbations to ER due to inconsistent testing equipment conditions, a modified stimulating-sensing configuration, graphically represented by the ER sensing map 1000 of FIG. 10, can be generated. According to the ER sensing map 1000, ERs are sensed from the “nearest neighbor” off-diagonal electrodes via up to four sense channels each comprising respective sense amplifiers and other signal conditioning components. A dedicated sense channel (CH4) can be electrically coupled to a specified set of two or more sensing electrodes to consistently sense ERs therefrom in response to evoking stimulation through one of stimulating electrodes. The two or more sensing electrodes may be ganged (electrically shorted to each other to function as one common electrode) and coupled to the dedicated channel CH4. As illustrated in FIG. 10, when evoking stimulation is delivered through any one of T1, T2a, T2b, or T2c, the segmented electrodes on the same level (T3a, T3b, and T3c) are ganged (electrically shorted) together, from which ERs are sensed using a common sense channel CH4. The stimulating-sensing configuration 1030A may ensure a consistent sense hardware condition (e.g., the sense amplifier and the tissue-electrode interface) being maintained at T3a, T3b, and T3c during different evoking tests at T1, T2a, T2b, and T2c. Similarly, when evoking stimulation is delivered through any one of T4, T3a, T3b, or T3c, the segmented electrodes on the same level T2a, T2b, and T2c may be ganged (electrically shorted) together, and the ERs may be sensed therefrom using the same sense channel CH4. The stimulating-sensing configuration 1030B may ensure a consistent sense hardware condition (e.g., the sense amplifier and the tissue-electrode interface) being maintained at T2a, T2b, and T2c during different evoking tests at evoking tests at T4, T3a, T3b, and T3c. By keeping the sense hardware condition consistent, a subset of off-diagonal electrodes in the stimulating-sensing configuration 1030A and 1030B may be used to correct sensing specific confounding factors.

The sensing circuit 710 may sense ERs in accordance with an ER sampling routine, such as determined or updated by the ER sampling routine circuit 726. The ER sampling routine includes timings of, or an order of executing, multiple stimulation and ER collection tests via respective stimulating and sensing electrodes on at least one lead in accordance with a stimulation-sensing configuration such as one of those shown in FIGS. 8-10. An improved ER sampling routine (including improved timing or order of executing multiple stimulation and ER collection tests), as will be described further below with respect to the ER sampling routine circuit 726, may help reduce, minimize, or correct perturbations introduced by confounding factors such as varying patient conditions and difference in testing equipment conditions.

The controller circuit 720 can include circuit sets comprising one or more other circuits or sub-circuits, such as a signal processor 722 and a therapy controller 728. The signal processor 722 may further include a filter circuit 724, an ER quality check circuit 725, an ER sampling routine circuit 726, and an ER feature extraction circuit 727. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

In various examples, portions of the functions of the controller circuit 720 may be implemented as a part of a microprocessor circuit. The microprocessor circuit can be a dedicated processor such as a digital signal processor, application specific integrated circuit (ASIC), microprocessor, or other type of processor for processing information including physical activity information. Alternatively, the microprocessor circuit can be a general purpose processor that can receive and execute a set of instructions of performing the methods or techniques described herein.

The filter circuit 724 may include a filter or a filter bank to filter the recorded ER signals. The signal analyzer circuit 725 may extract a signal feature from the filtered ER signal. Examples of the ER features can include a signal amplitude, magnitude, peak value, value range, a signal curve length, or a signal power or RMS value of an ER signal within a time window, such as the epoch-averaged ERs. The signal amplitude range or value range, also referred to as a peak-to-peak (P2P) value, can be measured as a difference between a maximum value or a minimum value of a dominant peak in the sensed evoked response or an epoch-averaged evoked response within the time window (also referred to as “max P2P” amplitude). Alternatively, the P2P value may be measured as a difference between a negative peak (trough) and an immediate next positive peak (also referred to as “N1-P2 P2P” amplitude). The signal curve length can be measured as accumulated signal value differences of the sensed evoked response (or an epoch-averaged evoked response) over consecutive unit times (e.g., consecutive data sampling intervals) within the time window. The signal power can be measured as an area under the curve (AUC) of the sensed evoked response (or the epoch-averaged evoked response) within the time window. In some examples, the signal analyzer circuit 725 may generate a spatial distribution of extracted signal features across the sensing locations of the sensing electrodes.

The ER quality check circuit 725 may perform a quality check of the collected and filtered ERs, including determining whether each of the collected and filtered ERs sensed from a group of sensing electrodes falls within a specified ER value range. ERs that fall outside of the specified ER value range fail the quality check, and are recognized as unsuccessful or unexpected measurements. In some examples, the quality check of the collected ERs may include a determination of whether the ERs respectively sensed from the group of sensing electrodes each satisfy a signal-to-noise ratio (SNR). In another example, the quality check may include a determination of whether the ERs respectively sensed from the group of sensing electrodes saturate the sense amplifier (thereby resulting in constant, saturation output).

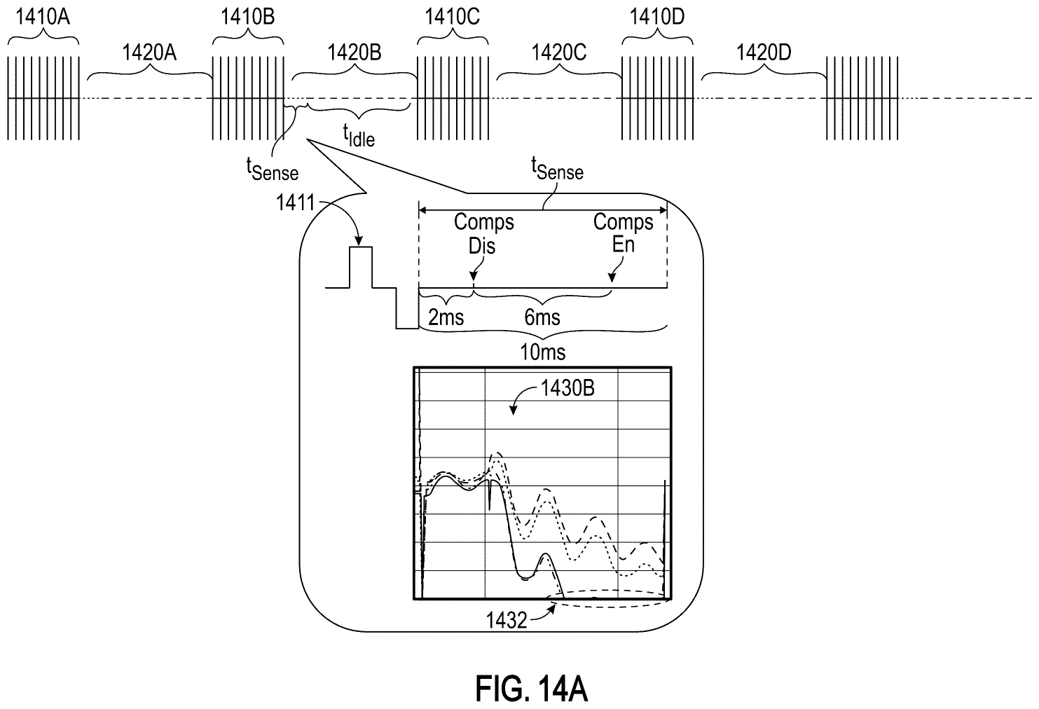

In an example, the ER value range for ER quality check may be determined based on a statistical distribution model of the ERs sensed at each of the group of sensing electrodes in response to evoking stimulation applied to a particular stimulating electrode in accordance with a stimulation-sensing configuration. Various statistical distribution models may be used. FIGS. 13A-13B illustrate by way of example and not limitation Gaussian models of ERs sensed from respective sensing electrodes in response to stimulation through stimulating electrode T1, and quality check of the ERs based on one or more model parameters. In particular, FIG. 13A illustrates a first Gaussian model 1310 representing a statistical distribution of ERs sensed at T2a, and a second Gaussian model 1320 representing a statistical distribution of ERs sensed at T3b. FIG. 13B illustrates an acceptable ER value range defined based on the mean (expected value) and the variance (or standard deviation, or SD) of the Gaussian distribution model. In an example, the acceptable ER value range can be defined between a lower bound (LB) 1332, computed as LB=mean−k*SD, and an upper bound (UB) 1334, computed as UB=mean+k*SD, where k is a constant (e.g., k=2 in one example). In an example, a Z-score may be calculated for an ER measurement as (ER−mean)/SD. The Z-score thus computed (or other statistical metrics) may be compared to a threshold value. The ER measurement is deemed to pass the quality check if the corresponding Z-score falls within a specific range (e.g., between −2 and 2 in an example), and to fail the quality check if the Z-score is outside of the range.

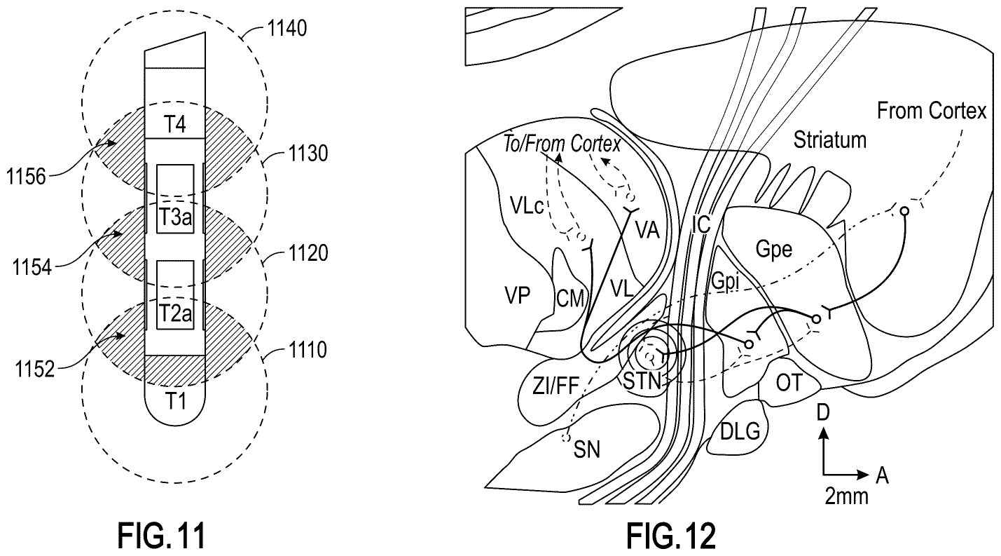

Referring back to FIG. 7, results of the quality check of the collected ERs may be provided to the ER sampling routine circuit 726 to determine or update an ER sampling routine to be used in the rest of the spatial survey subsequent to the stimulation through the first stimulating electrode. The ER sampling routine includes timings of, or an order of executing, multiple stimulation and ER collection tests via respective stimulating and sensing electrodes in accordance with a stimulation-sensing configuration such as one of those shown in FIGS. 8-10. As stated above, evoking stimulation applied at a stimulation site may produce residual effects on sensing locations particularly those proximate to the stimulation site, and cause perturbation on the ERs to stimulation delivered at another stimulation site. The residual effect may be due to a spatial overlap of stimulation fields produced by stimulations at adjacent stimulation sites. FIG. 11 illustrates an example of overlapping stimulation fields produced by stimulations through different stimulating electrodes on a lead, such as an overlapping region 1152 between a first stimulation field 1110 produced by stimulation through T1 and a second stimulation field 1120 produced by stimulation through T2a, an overlapping region 1154 between the second stimulation field 1120 and a third stimulation field 1130 produced by stimulation through T3a, and an overlapping region 1156 between the third stimulation field 1130 and a fourth stimulation field 1140 produced by stimulation through T4.

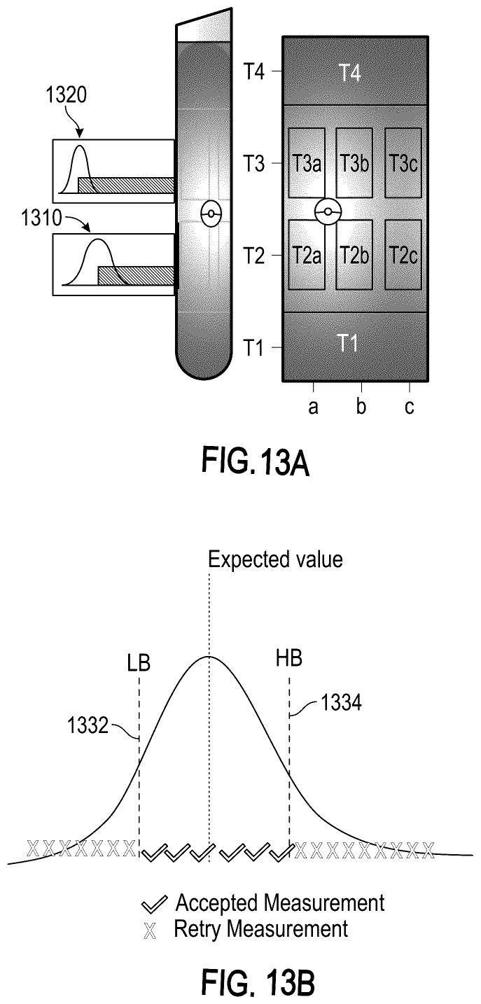

The residual effect may additionally or alternatively be caused by an anatomical overlap between neural structures, which can be concurrently evoked by stimulation at a stimulation site. In the context of DBS involving brain targets (e.g., STN and GPi) for managing Parkinson's Disease, anatomy of neural circuit elements by which the basal ganglia are connected to regions of the thalamus and cortex is important for an understanding of the mechanisms that are involved in ER generation responsive to DBS at neural targets such as STN and GPi. FIG. 12 illustrates an example of anatomical overlap between neural structures in a parasagittal slice through the nonhuman primate brain. The figure, reproduced from Devergnas and Wichmann Frontiers in systems neuroscience (2011) 5, 30, shows the major anatomical pathways involved in subthalamic nucleus (STN) DBS, including the internal capsule (IC), globus pallidus internus (GPi), and globus pallidus externus (GPe). As shown in FIG. 12, STN is located in a very crowded region of the brain. The STN is part of the “indirect” pathway of the basal ganglia. This pathway links the principal input structure of the basal ganglia, the striatum, to the output structures, the GPi, and the substantia nigra pars reticulata (SNr), via the external segment of the GPe and the STN. During DBS procedures, at least one lead can be implanted into the brain such that certain electrodes are proximal to brain regions like the STN. Typically, STN DBS electrodes are implanted into the central STN. The most ventral electrode tends to be implanted at the ventral border of the STN, or may extend into the dorsal SNr. Depending on the contact separation of the specific electrode used, the top contact is either located in the zona incerta (ZI) or in the central thalamus. Subsequently, continuous high-frequency stimulation is delivered via an IPG which can be externally programmed. Due to the anatomical network overlap, each time an anatomical target is engaged by stimulation, it is perturbed; and the perturbation can move the anatomical target out of its initial state, thereby affecting the ERs sensed therefrom.

The ER sampling routine may include a post-stimulation time delay for initiating a subsequent stimulation and ER collection test involving a second stimulating electrode (E2) different than the first stimulating electrode (E1). In an example, the post-stimulation time delay may be inversely related to a spatial distance between the stimulating electrodes E1 and E2, such that a stimulating electrode farther away from the first stimulating electrode E1 is engaged in stimulation prior to a stimulating electrode closer to the first stimulating electrode E1. The ER sampling routine may additionally or alternatively include an order of executing subsequent stimulation and ER collection tests involving respective stimulating electrodes different than E1. In an example, the order of executing subsequent multiple stimulation and ER collection tests may be inversely related to spatial distances between the respective stimulating electrodes and the first stimulating electrode E1. For example, in reference to FIGS. 9 and 10, after a first stimulation-ER collection test involving stimulation through stimulating electrode T1 and collection of ERs from sensing electrodes (e.g., T2a, T2b, and T2c) according to a stimulating-sensing configuration defined by the ER sensing map 900 or 1000, a test involving stimulation through any of T2a-T2c commences after a post-T1 delay Δt₁₂, a test involving stimulation through any of T3a-T3c commences after a post-T1 stimulation delay Δt₁₃, and a test involving stimulation through T4 may occur after a post-T1 stimulation delay Δt₁₄. The post-T1 delays may be determined such that Δt₁₃ is shorter than Δt₁₂ because any of T3a-T3c is farther away from T1 than any of T2a-T2c, and Δt₁₄ is shorter than Δt₁₃ because T4 is farther away from T1 than any of T3a-T3c.

The post-stimulation time delay thus determined may also be used to define an evoking order subsequent to the stimulation through T1. In the above example, because post-T1 delay Δt₁₄ for stimulation through T4 is shorter than Δt₁₃ for stimulation through any of T3a-T3c, which is shorter than Δt₁₂ for stimulation through any of T2a-T2c, a proper evoking order (and the order for executing corresponding stimulation and ER collection tests) may be determined as T1-T4-T3a (or T3b or T3c)-T2a (or T2b or T2c). Because the residual effects from stimulation is generally more prominent at sensing locations closer the stimulation site, the post-stimulation delay and/or the evoking order that are inversely related to the inter-stimulating electrode distance can reduce or minimize perturbation to ERs due to residual effects on sensing locations proximate the stimulation site.

The signal processor 722 may sequentially execute the multiple stimulation-ER collection tests in accordance with the determined or updated ER sampling routine (e.g., the post-stimulation time delays or the evoking order as described above). ERs collected from each of the multiple tests may be quality-checked by the ER quality check circuit 725. If one or a specified number of ERs in a test fail the quality check, the test may be recognized as a “failed test,” and an attempt to repeat such failed test can be performed. The repeating test may be scheduled at a specific time. In one example, the EP sampling routine circuit 726 may adjust the test order by appending the failed test to the end of the list, and repeating the failed test after executing every other of the multiple stimulation and ER collection tests in the list. In another example, the EP sampling routine circuit 726 may repeat the failed test prior to executing any other of the multiple stimulation and ER collection tests in the list. In some examples, the failed test may be repeated for up to a maximum number (N) of attempts, where N can be determined based on a change in brain state, or received as a user input.

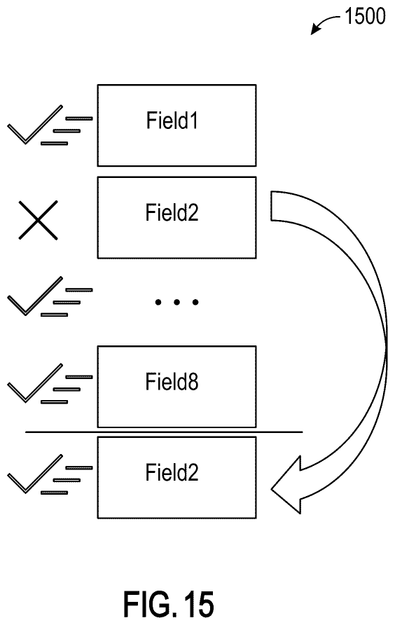

An example of scheduled repeat of the failed test is illustrated in FIG. 15. The diagram 1500 illustrates stimulation fields produced by each of eight stimulation-ER collection tests that involves one of the stimulating electrodes selected from T1, T2a-T2b, T3a-T3b, and T4. The tests are sequentially executed during a spatial survey process. A stimulation field can be established by stimulation through respective stimulating electrodes during each test (corresponding to Field 1 to Field 8), and ERs can be sensed from respective groups of sensing electrodes according to the stimulation-sensing configuration such as one of those as illustrated in FIGS. 8-10. In the example illustrated in FIG. 15, one or a specific number of ERs obtained from a test associated with Field 2 fail the quality check. Such failed test can be appended to the end of the list, and an attempt to repeat the failed test can be made after the test associated with Field 8.

The multiple stimulation and ER collection tests in accordance with the determined or updated ER sampling routine may be executed by one or more components of a neuromodulation system, such as one of IPG 102, RC 103, CP 104, or ETM 105 as illustrated in FIG. 1. In one example, at least a portion of the controller circuit 720, including the ER quality check circuit 725, the ER sampling routine circuit 726, and controlled execution of multiple stimulation and ER collection tests (and repeated testing of the failed stimulation-ER collection test), may be implemented as machine executable instructions in the firmware of an ambulatory medical device such as the IPG 102. The firmware may step through a list of stimulation and ER collection tests, deliver evoking stimulation and collecting ERs in accordance with the stimulation-sensing configuration, evaluate the quality of the collected ERs, identify a failed test, and retest the failed test at a scheduled time (e.g., after executing every other of the multiple stimulation and ER collection tests, or prior to executing any other of the multiple stimulation and ER collection tests). In another example, at least a portion of the controller circuit 720, including the ER quality check circuit 725, the ER sampling routine circuit 726, and controlled execution of multiple stimulation and ER collection tests (and repeated testing of the failed stimulation-ER collection test), may be implemented as machine executable instructions in the software of a programming device such as CP 104. The software may step through a list of stimulation and ER collection tests, passes each test to the firmware of the IPG 102 to evoke stimulation and to collect ERs in accordance with the stimulation-sensing configuration. The collected ERs may then be passed back to the software of CP 104. The software can evaluate the quality of the collected ERs, identify a failed test, and retest the failed test at a scheduled time (e.g., after executing every other of the multiple stimulation and ER collection tests, or prior to executing any other of the multiple stimulation and ER collection tests).

When the collected ERs pass the quality check, the ER feature extraction circuit 727 may extract one or more features from the collected ERs, and determine whether the ER features satisfy an acceptance criteria, such as matching a desired or target ER characteristic. The extracted ER features may include intensity features, temporal features, or signal morphological features. In an example, the ER features may be fit into one or more ER distribution models representing a spatial distribution of the ER features across the sensing electrodes such as selected in accordance with any one of the ER sensing maps 800, 900, or 1000, as illustrated in FIGS. 8-10, respectively. The ER distribution model may include a parametric model (e.g., a Gaussian distribution model, a periodic or wrapped Gaussian distribution model, an exponential distribution model, a Poisson distribution model, a Weibull distribution, among others), a regression model (e.g., a linear regression or a logistic regression model), or a non-parametric model (e.g., a decision tree, a K-nearest neighbor model, a support vector machine, or artificial neural network, among other machine-learning models). One or more model features may be derived from the fitted distribution model. In an example where the ER features are fitted to a Gaussian model, the model features may include one or more of the mean value or the standard deviation of the ER features. In another example, the model features may include a morphological or statistical feature of the fitted distribution model, such as an amplitude, a spatial location, or a width of a peak of the fitted model within a range defined by the plurality of sensing locations. In another example, the model features may include one or more of a positive peak amplitude (or a local maximum) or a negative peak amplitude (or a local minimum) of the fitted model within a range defined by the plurality of sensing locations. In yet another example, the model features may include a composite feature, such as a ratio of a positive peak amplitude to a negative peak amplitude of the fitted model within a range defined by the plurality of sensing locations. The ER distribution may be presented to the user on the user interface 750. In an example, the ER distribution may be depicted as a two dimensional (2D) hotspot view, such as a colored or grayscale heatmap. The “hotspot” represents the distribution center with a peak amplitude at a peak location.

The ER features generated by the ER feature extraction circuit 727 may be compared against one or more acceptance criteria 732 stored in the storage device 730 to determine whether a match to the desired or target response can be found. In some examples, the acceptance criteria are set, modulated, inspected, accepted by the clinical user, including ahead of or during operation. In some examples, the sensed ERs obtained in multiple stimulation-ER recording tests may be accumulated during which stimulation pulses are delivered via a particular stimulating electrode with varying stimulation parameter settings (e.g., stimulation amplitude, frequency, or pulse width). The ER features produced by the ER feature extraction circuit 727 may be determined using the accumulated ERs, and compared to the acceptance criteria 732. The acceptance criteria 732 may be provided by a user such as via the user interface 750. Alternatively, the acceptance criteria 732 may be predetermined and stored in the storage device 730. In an example, the acceptance criteria 732 is a user-provided acceptance bounds (e.g., upper and lower bounds, location bounds, properties bounds such presence, absence, or value of a feature) of the model features. In another example, the acceptance criteria includes a target ER distribution template representing a patient-specific ER distribution or a population-based ER distribution. In an example, the target ER template may be selected to relieve symptoms or for other goals such as lead placement for disease-modifying therapy or co-therapy (e.g., leads that inject drugs or light), and side-effect avoidance. In some examples, the acceptance criteria may include one of a plurality of candidate ER templates indexed by region, clinical institution, group, participants information, implanter information, or symptom relief goals, and a target ER template can be selected from the plurality of candidate ER templates based at least in part on one or more of an identification of an institution where the patient is implanted or treated with the electrostimulation, a group, or the participants information, an identification of an implanter that implants the lead, or the sensed indication of symptom relief of the patient.

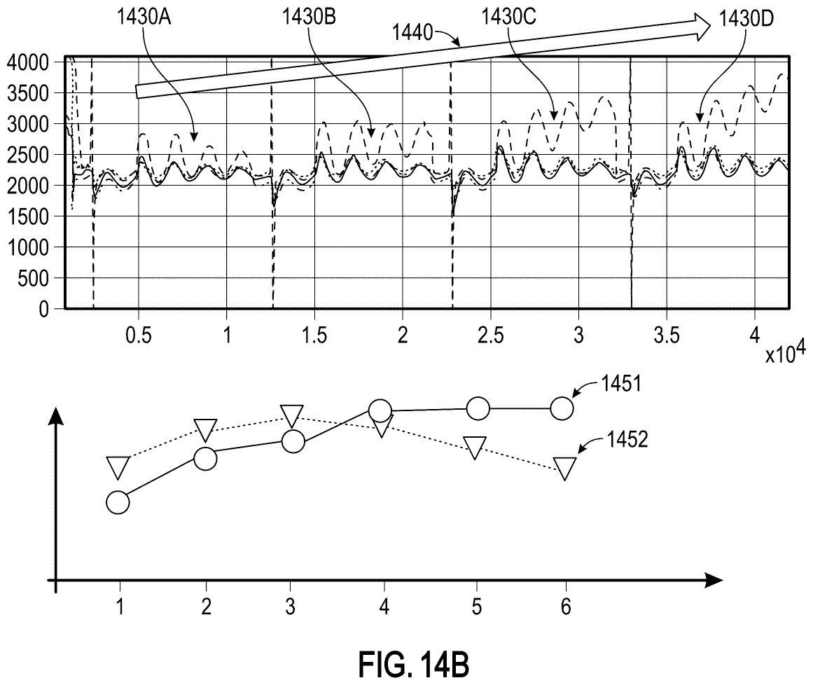

In various examples, the ER quality check circuit 725 may perform ER quality check at different time scales with respect to the evoking electrostimulation. Referring to FIG. 14A, the diagram therein illustrates electrostimulation and ER sensing during a spatial survey process, where stimulation bursts 1410A, 1410B, 1410C, 1410D, etc., are delivered to the neural target of interest such as via a stimulation electrode, and ERs are sensed during inter-burst intervals 1420A, 1420B, 1420C, 1420D, etc. via sensing electrodes determined in accordance with a stimulating-sensing electrode configuration such as one of those shown in FIGS. 8-10. During each ER epoch, ERs may be sensed during a specified ER sense window having a duration t_{Sense} defined within an inter-burst interval. The ER sense window may begin right after the last stimulation pulse of a burst (e.g., the last pulse 1411 of burst 1410B). By way of example and as illustrated, the inter-burst interval is 100 milliseconds (ms) in duration, including a t_{Sense} of 10 ms, followed by an idle period t_{Idle} of 90 ms. In one example, ER quality check can be performed at a relatively small timescale (e.g., millisecond-scale) such as within one individual epoch following a stimulation burst. In the illustrated example, following an individual stimulation burst 1410B, a collection of ERs 1430B can be sensed in accordance with different sensing configurations, each analyzed against a quality criterion, such as a saturation check to determine whether and/or when any of the artifacts or sensed ER saturates the sense amplifier. If one or more recordings or ERs fail the quality check (e.g., leading to saturation 1432), then the ER sampling routine circuit 726 can schedule a retest of the failed ER sensing configurations, or initiate one or more corrective actions such as changing the ER sensing configuration, adjusting sense amplifier parameters (e.g., gain adjustment, sense window adjustment, offset method adjustment (such as choosing a different offset management technique, such as advancing from passive to active measures, and advancing from firmware-free to firmware-in-the-loop methods), offset compensation current strength, timings of turning off or disabling the offset compensation or turning on or enabling the offset compensation (“CompDis” and “CompEn” respectively, as shown in FIG. 14A), or adjusting a stimulation parameter within the burst (e.g., pulse amplitude, pulse width, pulse rate or frequency). In case of biphasic stimulation where the stimulation pulse includes an active discharging phase such as the stimulation pulse 1411, the corrective actions may additionally or alternatively include adjusting a discharge parameter (e.g., discharge pulse duration, amplitude, timing, or trimming). Any of the stimulation parameters may be adjusted by altering the bits that control such parameters, among other approaches. In some examples, ER quality check may be further performed on responses after each pulse in a burst, such as by comparing a measured parameter (in response to the pulse delivered) to a quality criterion to determine whether or how good the ER is. The failed ER test (or a portion thereof) may be repeated using the adjusted parameters.

In addition or alternative to the ER quality check at the “fine” timescales and corresponding corrective actions that may be taken with respect to the failed ER test, in some examples, the ER quality check circuit 725 may perform ER quality check at a larger timescale (e.g., over several tens or hundreds of milliseconds or seconds), such as over multiple epochs following respective stimulation bursts. FIG. 14B illustrates by way of example ERs 1430A, 1430B, 1430C, 1430D, etc., each collected following respective stimulation bursts 1410A, 1410B, 1410C, 1410D, etc. Each of the ERs 1430A-1430D includes a collection of ERs sensed in accordance with different sensing configurations, and can be analyzed against a quality criterion, such as a saturation of amplifiers as described above, a mismatch between two serial ER measurements post respective stimulation bursts (which may be due to medication or activity or other slow variations in patient conditions), or a drift of ER measurement or ER feature values over multiple epochs. In the illustrated example, ERs sensed under certain sensing configuration demonstrate a baseline drift 1440 over multiple epochs.

To better detect and characterize the ER drift, the ER quality check circuit 725 may trend ER measurement or ER feature values over multiple epochs. FIG. 14B illustrates by way of example two ER trajectories 1451 and 1452 each representing ER measurements or ER feature values obtained from consecutive epochs (e.g., epoch 1 through epoch 6 as shown) following respective stimulation bursts. The ER quality check circuit 725 may perform quality check on the ER trajectories (e.g., a trend or shape of ER change over time) against a predetermined acceptance criterion. In the illustrated example, trajectory 1451A, characterized by an increase in ER feature value until reaching a plateau over multiple epochs, may be regarded as a desired or acceptable trajectory because it is generally expected that a responsive neural circuit, when repeatedly evoked with multiple stimulation bursts, would show a build-up of the evoked neural activities until reaching a plateau. In contrast, trajectory 1452, characterized by an initial increase in ER feature value and a subsequent immediate decrease without reaching a plateau, may be undesired or unacceptable as it could indicate for example an over-excitation of the same neural circuit by repeated bursts without adequate recovery, which could indicate a change in the state of the neural circuit or a move away from a prior baseline, which may adversely impact subsequent ER measurements from such neural circuit if the intention is to measure the same state.

The ER trajectory-based quality check as described above may provide guidance to corrective actions such as strategies for parameter adjustment. If one or more ERs fail the quality check (e.g., saturation or baseline drift), then the ER sampling routine circuit 726 may schedule a retest for the failed ER sensing configurations, or initiate other corrective actions such as changing the ER sensing configuration, adjusting sense amplifier parameters, adjusting a stimulation parameter, or adjusting a discharge parameter, and retest the failed ER test, as stated above. Alternatively, recordings might be marked or logged such that a user could repeat a test.

The ER quality check and corresponding corrective actions (such as parameter adjustment, in case of a failed ER test) as described above can be similarly performed in an even larger time scale, such as in minutes, hours, days, weeks, or months, which can be programmed under different patient conditions to meet individual patient needs. The parameter adjustment, when needed, can be dependent upon the quality check criteria and types of failed ER test (e.g., saturation, or baseline drift). In certain examples, the parameter adjustment may be dependent upon when the failed test occurs. For example, different parameter adjustment strategies may be taken in case of failed ER test within the first epoch versus failed ER test in later epochs after several bursts having been delivered.

The therapy controller 728 can generate a control signal to the electrostimulator 740 to adjust the neuromodulation therapy based on the ER features that satisfy the acceptance criteria 732. The electrostimulator 740 may be configured to deliver electrical stimulation according to a stimulation setting. The electrical stimulation may be delivered using a monopolar (far-field) or a bipolar (near-field) configuration. Examples of the therapy setting may include, electrode selection and configuration, stimulation parameter values including, for example, amplitudes, pulse width, frequency, pulse waveform, active or passive recharge mode, ON time, OFF time, therapy duration, and fractionalization, among others. In an example, the therapy controller 728 can be implemented as a proportional integral (PI) controller, a proportional-integral-derivative (PID) controller, or other suitable controller that takes the comparison of the sense ERs (or features or a distribution of the features thereof) to the acceptance criteria 732 as a feedback on the adjustment of stimulation settings. The types of data, and the recordings used to produce them, may vary regarding the type of acceptance criteria and operations employed. One ER measurement may be used to inform lead positioning (e.g., by sweeping a non-therapeutic sampling pulse across the space of the lead electrodes), another ER measurement may be used to determine or update a stimulation parameter (e.g., by sweeping a therapeutic sampling pulse across amplitudes).

The electrostimulator 740 can be an implantable module, such as incorporated within the IPG 10. Alternatively, the electrostimulator 740 can be an external stimulation device, such as incorporated with the ETS 40. In some examples, the user can choose to either send a notification (e.g., to the RC 45 or a smartphone with the patient) for a therapy reminder, or to automatically initiate or adjust neuromodulation therapy in accordance with the adjusted therapy setting. If an automatic therapy initiation is selected, the electrostimulator 740 can deliver stimulation in accordance with the adjusted therapy setting.

In some examples, the therapy controller 728 can generate a recommendation to the user to reposition the lead or to set or adjust the device setting (e.g., a programmable parameter of the electrostimulator 740). The repositioning of the lead or the adjustment of the device setting can cause the sensed ERs to align or more favorably compare to the acceptance criteria (e.g., an ER template) during an implantation procedure. In some examples, the therapy controller 728 may determine or modify therapeutic stimulation settings based on the sense ERs or features or a distribution of the features thereof. The electrostimulator 740 may deliver therapeutic stimulation (e.g., DBS) in accordance with the determined or modified therapeutic stimulation settings.

In some embodiments the display may provide a suggestion to the user to adjust the ER sampling routine or one or more stimulation parameters to cause the developed ER features to more favorably compare to the acceptance criteria 732 (e.g., an ER template). The recommendation can be displayed on the user interface 750. The user interface 750 can be a portable (e.g., handheld) device, such as the RC 45 or a smartphone (with executable software application) operable by the patient at his or her home without requiring extra clinic visits or consultation with a device expert. In another example, the user interface 750 can be a programmer device, such as the CP 50. In addition to the recommendation for lead replacement, other information may be displayed on the user interface 750 including, by way of example and not limitation, one or more of the sensed ERs, ER features, distribution of ER features, the acceptance criteria (e.g., one or more ER templates), or the comparison between the extracted ER features and the acceptance criteria. Further details of using ERs or ER features to optimize lead placement and/or a stimulation setting are described in commonly owned U.S. Provisional Patent Application No. 63/529,959, entitled “Evoked Response-Guided Neuromodulation Lead Placement” and filed on Jul. 31, 2023, the description of which is hereby incorporated by reference in their entirety.

In some examples, the user interface 750 may be configured to allow a physician to remotely review therapy settings and treatment history, consult with the patient to obtain information including pain relief and SCS-related side effects or symptoms, perform remote programming of the electrostimulator 740, or provide other treatment options to the patient. In an example, the user interface 750 may be configured to allow a user (e.g., the patient, the physician managing the patient, or a device expert) to view, program, or modify a device setting. For example, the user may use one or more user interface (UI) control elements to provide or adjust values of one or more device parameters, or select from a plurality of pre-defined stimulation programs for future use. In some examples, the user interface 750 can include a display to display textually or graphically information provided by the user via an input unit, and device settings including, for example, feature selection, sensing configurations, signal pre-processing settings, therapy settings, optionally with any intermediate calculations. In an example, the user interface 750 may present to the user an “optimal” or improved therapy setting, such as determined based on a closed-loop or adaptive feedback control of electrostimulation based on a selected evoked response signal feature, in accordance with various embodiments discussed in this document. In some examples, the user can use the user interface 750 to provide feedback on a neuromodulation therapy, including, for example, side effects or symptoms arise or persist associated with the neurostimulation, or severity of the symptom or a side effect.

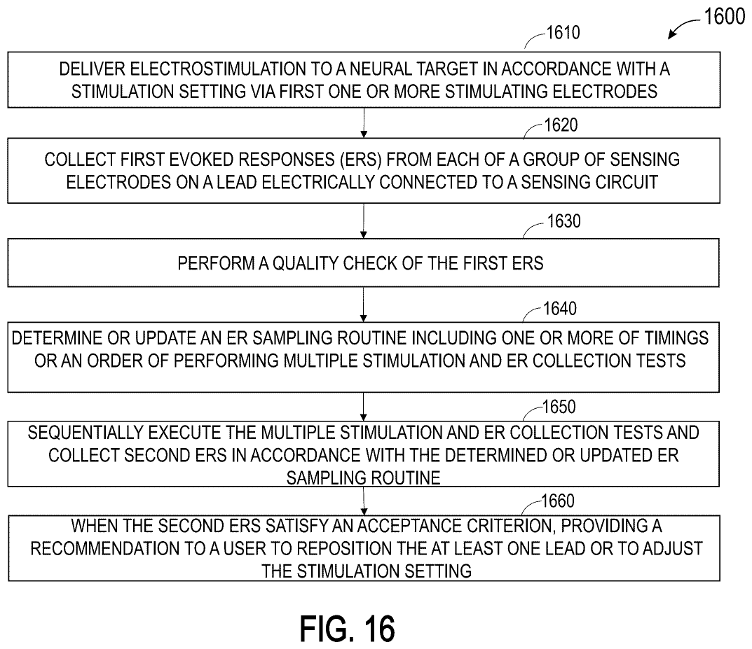

FIG. 16 is a flowchart illustrating an example method 1600 of collecting ERs and providing ER-based neurostimulation to a neural target of a patient. The method 1600 may be carried out using a medical system such as the neuromodulation system 700. In an example, the method 1600 may be implemented in a programmer device such as RC 45 or CP 50 in communication with an electrostimulator such as IPG 10 or electrostimulator 740. The method 1600 may be used to provide ER-based deep brain stimulation (DBS) at a brain target. The method 1600 may alternatively be used to provide ER-based neuromodulation therapy at other neural targets, such as spinal cord stimulation (SCS) at a spinal neural target.

At step 1610, electrostimulation may be delivered to a neural target in accordance with a stimulation setting via first one or more stimulating electrodes on at least one lead. At step 1620, a first set of evoked responses (ERs) to stimulation through the first one or more stimulating electrodes may be sensed from each of a group of sensing electrodes electrically connected to a sensing circuit, such as the sensing circuit 710. The group of sensing electrodes can be selected from the plurality of electrodes on the at least one lead and positioned at respective sensing locations. The ERs may be sensed in accordance with a stimulating-sensing electrode configuration, such as defined by any one of the ER sensing maps 800, 900, or 1000 as illustrated respectively in FIGS. 8-10. The stimulating-sensing electrode configuration may include an “on-diagonal” sensing configuration where the same electrode is used for delivering electrostimulation pulses and for recording an ER to that electrostimulation in the same stimulation session. The ER sensed therefrom is referred to as “on-diagonal” ER. The stimulating-sensing electrode configuration may also include an “off-diagonal” sensing configuration where different electrodes are used for delivering electrostimulation pulses and for recording an ER to that electrostimulation in the same stimulation session. The ER sensed therefrom is referred to as “off-diagonal” ER. In one example, the stimulating-sensing electrode configuration may include only the “off-diagonal” ERs, but not the “on-diagonal” ERs. Alternatively, as illustrated in FIG. 9, the stimulating-sensing electrode configuration may be a “nearest neighbor” configuration that includes a selected subset, less than an entirety, of available electrodes for ER sensing. The selected subset can be those non-diagonal electrodes within a specific proximity to the stimulating electrode being used for delivering electrostimulation pulses. The selective ERs from a subset of sensing electrodes as defined by the stimulating-sensing electrode configuration can improve the efficiency without compromising the accuracy of identifying ERs that match the desired or target response for different desired evoked response targets. Further, in some examples as illustrated in FIG. 10, the stimulating-sensing electrode configuration may be a modified version of the “nearest neighbor” configuration, where the “on-diagonal” ERs 1010 may be excluded from the ERs being used for guiding the lead placement and device programming, and the “off-diagonal” ERs 1020 from the “nearest neighbor” off-diagonal electrodes are sensed via up to four sense channels (each having respective sense amplifiers and other signal conditioning circuitry), including a dedicated sense channel electrically coupled to a specified set of two or more sensing electrodes to consistently sense ERs therefrom when evoking stimulation is applied to one of other electrodes. The set of two or more sensing electrodes may be ganged (electrically shorted to each other to function as one common electrode) and coupled to the dedicated channel. Such stimulating-sensing electrode configuration may help reduce or minimize the perturbations to ER due to inconsistent testing equipment conditions.

At step 1630, a quality check of the collected first set of ERs to stimulation through the first one or more stimulating electrodes may be performed to identify one or more unsuccessful or unexpected ERs from the first set of ERs, such as using the ER quality check circuit 725. The quality check may involve comparing the ERs respectively sensed from a group of sensing electrodes against a specified ER value range. ERs that fall outside of the specified ER value range are deemed unsuccessful or unexpected values that fail the quality check. In an example, the ER value range may be determined based on a statistical distribution model of the first set of the ERs sensed, such as a Gaussian distribution model, although other statistical models may be used. The quality check of the collected ERs may additionally or alternatively include a determination of whether each of the ERs respectively sensed from the group of sensing electrodes satisfies a signal-to-noise ratio (SNR).

At step 1640, an ER sampling routine may be determined or updated based on the result of quality check from step 1630. The ER sampling routine includes timings of, or an order of executing, multiple stimulation and ER collection tests (subsequent to the first evoking stimulation) via respective stimulating and sensing electrodes in accordance with a stimulation-sensing configuration, such as one of those shown in FIGS. 8-10. An optimal ER sampling routine may help reduce, minimize, or correct perturbations introduced by confounding factors such as varying patient conditions and difference in testing equipment conditions. In an example, a post-stimulation time delay for initiating a subsequent stimulation and ER collection test involving a second stimulating electrode (E2) different than the first evoking stimulating electrode (E1) may be inversely related to a distance between the E1 and E2 stimulating electrodes. In another example, an order of executing subsequent stimulation and ER collection tests involving respective stimulating electrodes different than the first stimulating electrode may be inversely related to spatial distances between the respective stimulating electrodes and the first stimulating electrode E1.

At step 1650, the multiple stimulation and ER collection tests may be sequentially executed in accordance with the determined or updated ER sampling routine produced at step 1640. ERs collected from each of the multiple tests (also referred to as “second ERs” to distinguish from the first set of ERs collected at step 1620 in response to stimulation through the first stimulating electrode) may be quality-checked by the ER quality check circuit 725. If one or a specified number of ERs in a test fail the quality check, the test is recognized as a “failed test,” and an attempt to repeat the “failed test” can be scheduled at a specific time, such as prior to executing any other of the multiple stimulation and ER collection tests in one example, or after executing every other of the multiple stimulation and ER collection tests in another example. The failed test may be repeated for up to a maximum number (N) of attempts.

At step 1660, when the ERs collected at step 1650 satisfy an acceptance criterion, a recommendation to reposition the at least one lead or to set or adjust the stimulation setting may be provided to a user. In an example, one or more ER features may be extracted from the collected ERs, which can be compared to the acceptance criterion, such as matching a desired or target ER characteristic. The comparison result can be displayed to the user on a user interface. Based on such comparison, a recommendation can be provided to the user to reposition the at least one lead, such as pushing, pulling, shifting, or rotating the lead to achieve a desired target response. The comparison result may additionally or alternatively be used to guide automatic adjustment of stimulation setting. During the repositioning of the at least one lead and/or the adjustment of stimulating setting, the ERs may be sensed, ER features and/or distributions may be determined, and comparison to the acceptance criteria can be updated in substantially real time and displayed to the user. The user may continuate repositioning the at least one lead and/or adjusting the stimulating setting until the sensed ERs compare more favorably to the acceptance criteria. The sense ERs or features or a distribution of the features generated therefrom may be used as feedback to modify therapeutic stimulation settings.

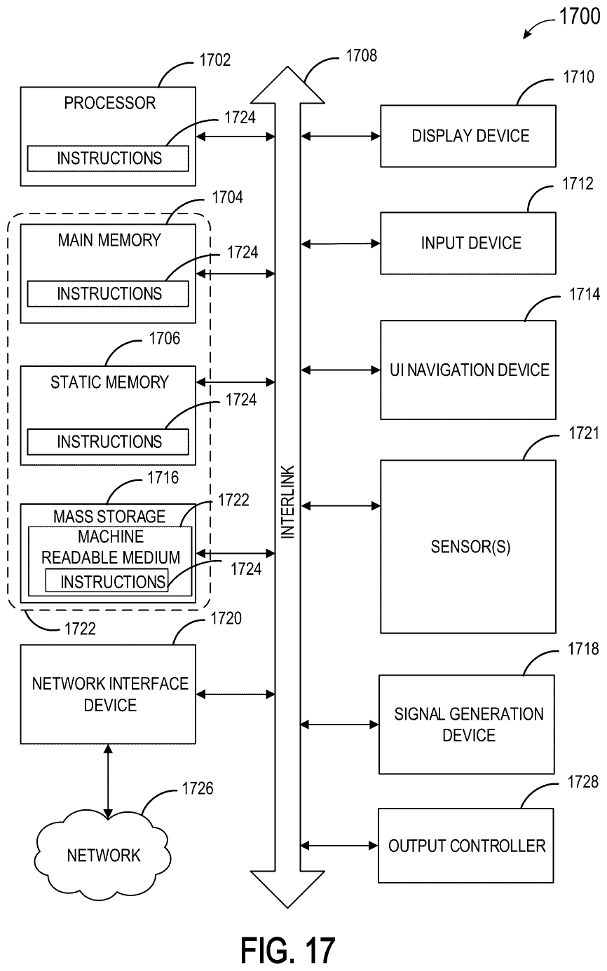

FIG. 17 illustrates generally a block diagram of an example machine 1700 upon which any one or more of the techniques (e.g., methodologies) discussed herein may perform. Portions of this description may apply to the computing framework of various portions of the neuromodulation device or the external programmer device.

In alternative examples, the machine 1700 may operate as a standalone device or may be connected (e.g., networked) to other machines. In a networked deployment, the machine 1700 may operate in the capacity of a server machine, a client machine, or both in server-client network environments. In an example, the machine 1700 may act as a peer machine in peer-to-peer (P2P) (or other distributed) network environment. The machine 1700 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), among other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 1700 may include a hardware processor 1702 (e.g., a central processing unit (CPU), a graphics processing unit (GPU), a hardware processor core, algorithm specific ASIC, or any combination thereof), a main memory 1704 and a static memory 1706, some or all of which may communicate with each other via an interlink (e.g., bus) 1708. The machine 1700 may further include a display unit 1710 (e.g., a raster display, vector display, holographic display, etc.), an alphanumeric input device 1712 (e.g., a keyboard), and a user interface (UI) navigation device 1714 (e.g., a mouse). In an example, the display unit 1710, input device 1712 and UI navigation device 1714 may be a touch screen display. The machine 1700 may additionally include a storage device (e.g., drive unit) 1716, a signal generation device 1718 (e.g., a speaker), a network interface device 1720, and one or more sensors 1721, such as a global positioning system (GPS) sensor, compass, accelerometer, or other sensors. The machine 1700 may include an output controller 1728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1716 may include a machine-readable medium 1722 on which is stored one or more sets of data structures or instructions 1724 (e.g., software) embodying or utilized by any one or more of the techniques or functions described herein. The instructions 1724 may also reside, completely or at least partially, within the main memory 1704, within static memory 1706, or within the hardware processor 1702 during execution thereof by the machine 1700. In an example, one or any combination of the hardware processor 1702, the main memory 1704, the static memory 1706, or the storage device 1716 may constitute machine readable media.

While the machine-readable medium 1722 is illustrated as a single medium, the term “machine readable medium” may include a single medium or multiple media (e.g., a centralized or distributed database, and/or associated caches and servers) configured to store the one or more instructions 1724.

The term “machine readable medium” may include any medium that is capable of storing, encoding, or carrying instructions for execution by the machine 1700 and that cause the machine 1700 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 1724 may further be transmitted or received over a communication network 1726 using a transmission medium via the network interface device 1720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 1720 may include one or more physical jacks (e.g., Ethernet, coaxial, or phone jacks) or one or more antennas to connect to the communication network 1726. In an example, the network interface device 1720 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1700, and includes digital or analog communications signals or other intangible medium to facilitate communication of such software.

Various examples are illustrated in the figures above. One or more features from one or more of these examples may be combined to form other examples.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A neuromodulation system, comprising:
an electrostimulator configured to provide electrostimulation to a neural target of a patient via one or more stimulating electrodes on at least one lead;
a sensing circuit configured to sense evoked responses (ERs) to electrostimulation via one or more sensing electrodes on the at least one lead; and
a controller circuit operably connected to the electrostimulator and the sensing circuit, the controller circuit configured to:
in response to electrostimulation of the neural target through first one or more stimulating electrodes in accordance with a stimulation setting, collect first ERs sensed from a group of sensing electrodes on the at least one lead;
perform a quality check of the first ERs;
based on a result of the quality check of the first ERs, determine or update an ER sampling routine including one or more of timings or an order of performing multiple stimulation and ER collection tests subsequent to the electrostimulation through the first one or more stimulating electrodes; and
sequentially execute the multiple stimulation and ER collection tests and collect second ERs in accordance with the determined or updated ER sampling routine.

2. The neuromodulation system of claim 1, wherein the controller circuit is configured to, when the second ERs satisfy an acceptance criterion, provide a recommendation to a user to reposition the at least one lead or to set or adjust the stimulation setting.

3. The neuromodulation system of claim 1, wherein to perform the quality check includes to determine that the first ERs pass the quality check when each of the first ERs falls within a specific ER value range, or fail the quality check when one or more of the ERs fall outside of the specific ER value range,
wherein the controller circuit is configured to determine the specific ER value range based on a statistical distribution model of the first ERs.

4. The neuromodulation system of claim 3, wherein, when one or more of the first ERs fail the quality check, the controller circuit is configured to determine or update the ER sampling routine including a post-stimulation time delay before initiating a subsequent stimulation and ER collection test involving stimulation through second one or more stimulating electrodes different than the first one or more stimulating electrodes,
wherein the post-stimulation time delay is inversely related to a spatial distance between the first and the second stimulating electrodes.

5. The neuromodulation system of claim 3, wherein, when one or more of the first ERs fail the quality check, the controller circuit is configured to determine or update the ER sampling routine including an order of executing the subsequent multiple stimulation and ER collection tests involving stimulations via respective stimulating electrodes different than the first one or more stimulating electrodes,
Wherein the order of executing the subsequent multiple stimulation and ER collection tests is inversely related to spatial distances between the respective stimulating electrodes and the first one or more stimulating electrodes.

6. The neuromodulation system of claim 1, wherein the electrostimulation of the neural target includes a plurality of stimulation bursts, and the first ERs include temporal ER portions corresponding to the plurality of stimulation bursts,
wherein the controller circuit is configured to perform the quality check of the first ERs at one or more distinct time scales with respect to the electrostimulation, including at least one of:
a first quality check of one of the temporal ER portions corresponding to an individual burst of the plurality of stimulation bursts; or
a second quality check of a plurality of the temporal ER portions corresponding to multiple distinct bursts of the plurality of stimulation bursts.

7. The neuromodulation system of claim 1, wherein the controller circuit is configured to:
perform the quality check of the second ERs;
identify, from the multiple stimulation and ER collection tests, at least one failed test with corresponding one or more ERs that fail the quality check; and
repeat the identified at least one failed test up to a specific maximum number of attempts.

8. The neuromodulation system of claim 7, wherein the controller circuit is configured to repeat the identified at least one test at a scheduled time, including:
to repeat the identified at least one failed test prior to executing any other of the multiple stimulation and ER collection tests; or
to append the identified at least one failed test to the end of the multiple stimulation and ER collection tests, and repeat the identified at least one test after executing every other of the multiple stimulation and ER collection tests.

9. The neuromodulation system of claim 1, comprising a programming device and an ambulatory medical device (AMD) communicatively coupled to the programming device, the AMD including at least a portion of the controller circuit configured to sequentially execute the multiple stimulation and ER collection tests and collect the second ERs.

10. The neuromodulation system of claim 1, comprising a programming device and an ambulatory medical device (AMD) communicatively coupled to the programming device, the programming device including at least a portion of the controller circuit configured to sequentially execute the multiple stimulation and ER collection tests and collect the second ERs.

11. The neuromodulation system of claim 1, wherein the sensing circuit includes a dedicated sensing channel electrically coupled to two or more sensing electrodes on the lead to concurrently sense ERs therefrom in response to electrostimulation through each of two or more distinct stimulation electrodes.

12. The neuromodulation system of claim 11, wherein the two or more sensing electrodes are at least temporarily electrically shorted to each other when coupled to the dedicated sensing channel, the dedicated sensing channel configured to sense the ERs from the electrically shorted electrodes.

13. A method of providing neurostimulation to a neural target of a patient via a neuromodulation system that comprises an electrostimulator and at least one lead coupled thereto, the method comprising:
delivering electrostimulation to the neural target in accordance with a stimulation setting via first one or more stimulating electrodes on the at least one lead;
collecting first evoked responses (ERs) from each of a group of sensing electrodes on the at least one lead via a sensing circuit;
performing a quality check of the first ERs;
based on a result of the quality check of the first ERs, determining or updating an ER sampling routine including one or more of timings or an order of performing multiple stimulation and ER collection tests subsequent to the electrostimulation through the first one or more stimulating electrodes;
sequentially executing the multiple stimulation and ER collection tests and collecting second ERs in accordance with the determined or updated ER sampling routine; and
when the second ERs satisfy an acceptance criterion, providing a recommendation to a user to reposition the at least one lead or to set or adjust the stimulation setting.

14. The method of claim 13, comprising determining an ER value range based on a statistical distribution model of the first ERs,
wherein performing the quality check includes determining that the first ERs pass the quality check when each of the first ERs falls within the determined ER value range, or fail the quality check when one or more of the ERs fall outside of the determined ER value range.

15. The method of claim 14, wherein determining or updating the ER sampling routine includes, when one or more of the first ERs fail the quality check, determining a post-stimulation time delay before initiating a subsequent multiple stimulation and ER collection test involving stimulation through second one or more stimulating electrodes different than the first one or more stimulating electrodes, the post-stimulation time delay inversely related to a spatial distance between the first and the second stimulating electrodes.

16. The method of claim 14, wherein determining or updating the ER sampling routine includes, when one or more of the first ERs fail the quality check, determine or update an order of executing the subsequent multiple stimulation and ER collection tests involving stimulations via respective stimulating electrodes different than the first one or more stimulating electrodes, the order of executing the subsequent multiple stimulation and ER collection tests inversely related to spatial distances between the respective stimulating electrodes and the first one or more stimulating electrodes.

17. The method of claim 13, comprising:
performing the quality check of the second ERs;
identifying, from the multiple stimulation and ER collection tests, at least one failed test with corresponding one or more ERs that fail the quality check; and
repeating the identified at least one failed test up to a specific maximum number of attempts.

18. The method of claim 17, wherein repeating the identified at least one test occurs at a scheduled time prior to executing any other of the multiple stimulation and ER collection tests, or after executing every other of the multiple stimulation and ER collection tests.

19. The method of claim 13, wherein the electrostimulation of the neural target includes a plurality of stimulation bursts, and the first ERs include temporal ER portions corresponding to the plurality of stimulation bursts,
wherein the quality check of the first ERs is performed at one or more distinct time scales with respect to the electrostimulation, including at least one of:
a first quality check of one of the temporal ER portions corresponding to an individual burst of the plurality of stimulation bursts; or
a second quality check of a plurality of the temporal ER portions corresponding to multiple distinct bursts of the plurality of stimulation bursts.

20. The method of claim 13, wherein the first or the second ERs include ERs to electrostimulation through each of two or more distinct stimulation electrodes, and sensed from two or more sensing electrodes at least temporarily electrically shorted to each other and coupled to a dedicated sensing channel.
